(19) United States
(12) Patent Application Publication
(10) Pub. No.: US 2025/0073284 A1
(43) Pub. Date: Mar. 6, 2025

(54) WEISSELLA CONFUSA, CULTURE METHOD THEREFOR AND USE THEREOF

(71) Applicant: SHANGHAI SINGEN PET NUTRITION CO., LTD., Shanghai (CN)

(72) Inventors: Jou-An SHIH, Shanghai (CN); Chun-Hui SHIH, Shanghai (CN); Ping-Hui SHIH, Shanghai (CN)

(73) Assignee: SHANGHAI SINGEN PET NUTRITION CO., LTD., Shanghai (CN)

(21) Appl. No.: 18/281,312

(22) PCT Filed: Mar. 2, 2022
(86) PCT No.: PCT/CN2022/078752

§ 371 Date: Sep. 11, 2023

(30) Foreign Application Priority Data
Sep. 28, 2021 (CN) 202111145446.X

(51) Int. Cl. A61K 35/744; A61P 1/12; C12N 1/20

(52) U.S. Cl. CPC A61K 35/744; A61P 1/12; C12N 1/20

(57) ABSTRACT

Weissella confusa WSG1 with digestion-assisting capacity is provided. The bacterial strain was preserved in the China General Microbiological Culture Collection Center (CGMCC) on Jun. 11, 2021 with a preservation number of CGMCC NO. 22697 and a nucleotide sequence shown in SEQ ID NO: 1. The bacterial strain of the present invention overcomes the problem of poor treatment effect of antibiotics and other medicaments in the prior art, has good safety, can survive in the gastrointestinal environment, has good susceptibility to antibiotics, has a remarkable inhibitory effect on common diarrhea-causing pathogenic bacteria, has digestion-assisting capacity, and is orally administered without toxicity. Therefore, a new available bacterial strain for the development of probiotics for maintaining the health capability of the intestinal tracts of canine organisms is provided herein.

### CROSS REFERENCE TO THE RELATED APPLICATIONS

This application is the national phase entry of International Application No. PCT/CN2022/078752, filed on Mar. 2, 2022, which is based upon and claims priority to Chinese Patent Application No. 202111145446.X, filed on Sep. 28, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBRZBC077-Sequence Lisint.txt, created on Aug. 2, 2023, and is 2,321 bytes in size.

### TECHNICAL FIELD

The present invention relates to the technical field of microorganisms, and in particular to Weissella confusa, a culture method therefor and use thereof.

### BACKGROUND

Before dogs are domesticated in a domestic environment, they consume various uncooked foods in the natural environment to supplement probiotics in their body. However, pet dogs have been out of the natural environment for a long time and are being kept at home, and if they eat processed foods such as dog food and canned food for a long time, they cannot supplement a proper amount of probiotics from these foods, and even the additional additives in these foods can destroy the colony balance in the pet dogs, so that the pet dogs will have the problems of diarrhea, vomiting and the like. Some dogs have immature digestive systems and weak digestive capacity, and diarrhea, vomiting and the like are more common in these dogs. In the prior art, medicaments such as antibiotics are commonly available for the treatment, such that pathogenic bacteria can generate drug resistance, and even the relapse of diseases occurs.

### SUMMARY

An objective of the present invention is to provide a probiotic strain capable of improving the digestive capacity of dogs and inhibiting common diarrhea-causing pathogenic bacteria, thus providing a new available bacterial strain for the development of probiotics for maintaining the health capability of the intestinal tracts of canine organisms.

In order to achieve the above objective, the present invention provides the following technical solutions: Weissella confusa is provided, which is

Weissella confusa WSG1 and preserved in the China General Microbiological Culture Collection Center (CGMCC) on Jun. 11, 2021 with a preservation number of CGMCC NO. 22697.

Preferably, a nucleotide sequence of 16S IRNA of the Weissella confusa is shown in SEQ ID NO: 1.

The present invention further provides a culture method for Weissella confusa, comprising inoculating the Weissella confusa WSG1 into a culture medium for culture, with an initial pH of the culture being 5.0 to 6.5, a temperature of the culture being 35° C. to 38° C., and a culture time being 15 h to 17 h; wherein the culture medium comprises a carbon source and a nitrogen source, and a content of the carbon source and a content of the nitrogen source in the culture medium are independently 1.0% to 3.0%.

Preferably, the carbon source is one or more of glucose, sucrose and starch. Preferably, the nitrogen source is one or more of peptone, beef extract and yeast powder.

The present invention further provides use of Weissella confusa for the manufacturing of a medicament for assisting digestion.

Preferably, the medicament is a veterinary medicament.

Preferably, the veterinary medicament is a canine-specific medicament.

The present invention further provides use of Weissella confusa for the manufacturing of a feed.

The present invention further provides use of Weissella confusa for the manufacturing of a medicament for treating diarrhea.

The present invention has the technical effects and advantages that:
the present invention provides Weissella confusa WSG1 with digestion-assisting capacity, which has been proved to be safe by hemolytic evaluation and susceptibility evaluation, can survive in the gastrointestinal environment, has good susceptibility to antibiotics, has a remarkable inhibitory effect on common diarrhea-causing pathogenic bacteria, has digestion-assisting capacity, and is orally administered without toxicity. Therefore, a new available bacterial strain for the development of probiotics for maintaining the health capability of the intestinal tracts of canine organisms is provided herein.

### BRIEF DESCRIPTION OF THE DRAWINGS

FIG. 1 shows a cover of an identification report of the Institute of Microbiology, Chinese Academy of Sciences;

FIG. 2 shows content 1 of the identification report of the Institute of Microbiology, Chinese Academy of Sciences;

FIG. 3 shows content 2 of the identification report of the Institute of Microbiology, Chinese Academy of Sciences; and

FIG. 4 shows a proteolytic ring produced by the strain WSG1. Preservation Description

Weissella confusa WSG1 is preserved in the China General Microbiological Culture Collection Center (CGMCC) with a preservation address of No. 3, Yard 1, Beichen West Road, Chaoyang District, Beijing, China, a preservation date of Jun. 11, 2021 and a preservation number of CGMCC NO. 22697.

### DETAILED DESCRIPTION OF THE EMBODIMENTS

The present invention provides Weissella confusa being Weissella confusa WSG1 and preserved in the China General Microbiological Culture Collection Center (CGMCC) on Jun. 11, 2021 with a preservation number of CGMCC NO. 22697. In the present invention, a nucleotide sequence of 16S rRNA of the Weissella confusa is preferably as shown in SEQ ID NO: 1.

The present invention further provides a culture method for Weissella confusa, comprising inoculating the Weissella confusa WSG1 into a culture medium for culture. In the present invention, an initial pH of the culture is preferably 5.0 to 6.5, more preferably 5.5 to 6.2, and still more preferably 5.8 to 6.0; a temperature of the culture is preferably 35° C. to 38° C., and more preferably 36° C. to 37° C.; a culture time is preferably 15 h to 17 h, more preferably 15.5 h to 16.5 h, and still more preferably 15.8 h to 16.2 h.

In the present invention, the culture medium preferably comprises a carbon source and a nitrogen source, wherein the carbon source is preferably one or more of glucose, sucrose and starch, and a content of the carbon source is preferably 1.0% to 3.0%, more preferably 1.5% to 2.5%, and still more preferably 1.7% to 2.0%; the nitrogen source is preferably one or more of peptone, beef extract and yeast powder, the peptone is preferably casein peptone, and a content of the nitrogen source is preferably 1.0% to 3.0%, more preferably 1.4% to 2.8%, and still more preferably 1.9% to 2.5%.

The present invention further provides use of Weissella confusa for the manufacturing of a medicament for assisting digestion, wherein the medicament is preferably a veterinary medicament, and more preferably a canine-specific medicament. In the present invention, the concentration of live bacteria of Weissella confusa in the medicament is 1×10⁸ CFU/kg to 6×10⁸ CFU/kg, more preferably 2×10⁸ CFU/kg to 5×10⁸ CFU/kg, and still more preferably 3×10⁸ CFU/kg to 4×10⁸ CFU/kg; the medicament preferably takes Weissella confusa as the only active ingredient; further preferably, the medicament further comprises other active ingredients for assisting digestion; the medicament comprises an excipient, wherein the type of the excipient is not specially limited herein, which may be a pharmaceutical excipient common in the art.

The present invention further provides use of Weissella confusa for the manufacturing of a feed, wherein the feed is preferably a canine feed, and more preferably a canine-specific feed. In the present invention, the concentration of live bacteria of Weissella confusa in the feed is 1×10⁸ CFU/kg to 6×10⁸ CFU/kg, more preferably 2×10⁸ CFU/kg to 5×10⁸ CFU/kg, and still more preferably 3×10⁸ CFU/kg to 4×10⁸ CFU/kg; the feed preferably takes Weissella confusa as the only active ingredient for assisting digestion; further preferably, the feed further comprises other active ingredients for assisting digestion; the feed comprises a nutritional component, wherein the type of the nutritional component is not specially limited herein, which may be a nutritional component common in the art.

The present invention further provides use of Weissella confusa for the manufacturing of a medicament for treating diarrhea, wherein the medicament is preferably a veterinary medicament, and more preferably a canine-specific medicament. In the present invention, the concentration of live bacteria of Weissella confusa in the medicament is 1×10⁸ CFU/kg to 6×10⁸ CFU/kg, more preferably 2×10⁸ CFU/kg to 5×10⁸ CFU/kg, and still more preferably 3×10⁸ CFU/kg to 4×10⁸ CFU/kg; the medicament preferably takes Weissella confusa as the only active ingredient; further preferably, the medicament further comprises other active ingredients for treating diarrhea; the medicament comprises an excipient, wherein the type of the excipient is not specially limited herein, which may be a pharmaceutical excipient common in the art.

The technical solutions provided by the present invention will be described in detail below with reference to the examples, which, however, should not be construed as limiting the scope of the present invention.

### Example 1

Weissella confusa WSG1 was separated from feces of puppies and was preserved in the China General Microbiological Culture Collection Center (CGMCC) on Jun. 11, 2021 with a preservation number of CGMCC NO. 22697; the nucleotide sequence of the Weissella confusa was shown in SEQ ID NO: 1.

The relevant performance assays were performed using MRS medium for culture. Example 2

The activated strain WSG1 (preservation number CGMCC NO. 22697) was inoculated into a culture medium in an inoculation amount of 2% and cultured for 16 h at 37° C.

The initial pH of the culture medium was 6.0;

the specific formula of the culture medium was as follows:
MRS medium:
Casein peptone 10.0 g;
Beef extract 10.0 g;
Yeast extract 5.0 g;
Glucose (C₆H₁₂O₆·H₂O) 20.0 g;
Sodium acetate (CH₃COONa·3H₂O) 5.0 g;
Diamine citrate [(NH₄)₂HC₆H₅O₇] 2.0 g;
Tween 80 1.0 g;
Dipotassium hydrogen phosphate (K₂HPO₄·3H₂O) 2.0 g;
Magnesium sulfate (MgSO₄ 7·H₂O) 0.58 g;
Manganese sulfate (MnSO₄·H₂O) 0.25 g;
Calcium carbonate 20.0 g;
Agar 18.0 g;
Distilled water 1000 mL

Live bacteria were counted using plate method to obtain a concentration of 5.52×10¹¹ CFU/mL.

### Experimental Example 1: Safety Evaluation-Hemolytic Evaluation

The experimental process was as follows: a fresh culture of the strain WSG1 was streaked and inoculated onto a solid culture medium containing 5% sheep blood, and whether there was a hemolysis or not was observed, and the strain which did not generate hemolysis was determined to be a safe strain.

The experimental results were as follows: there was no hemolysis generated.

### Experimental Example 2: Safety Evaluation-Susceptibility Evaluation

The experimental process was as follows: the susceptibility of the strain WSG1 to common antibiotics was researched using a K-B method and an MIC method, with the antibiotics including: vancomycin, chloramphenicol, cephalothin, amoxicillin, clindamycin, enrofloxacin, penicillin, cefalexin, cefradine, azithromycin, oxacillin, cefazolin, tetracycline, ciprofloxacin, and marbofloxacin (MIC method).

The experimental results were as follows: the diameters (mm) of the zones of inhibition and MIC values were shown in Tables 1 and 2.

TABLE 1
Diameters (mm) of zones of inhibition of strain WSG1 against antibiotics

| | Vancomycin | Chloramphenicol | Cephalothin | Amoxicillin | Clindamycin | Enrofloxacin | Penicillin |
|---|---|---|---|---|---|---|---|
| Diameter | 0.0 | 23.5 | 23.5 | 15.0 | 23.0 | 13.5 | 23.5 |
| of zone | | | | | | | |
| of | | | | | | | |
| inhibition | | | | | | | |
| | Cefradine | Azithromycin | Oxacillin | Cefazolin | Tetracycline | Ciprofloxacin | Cefalexin |
| Diameter | 17.5 | 15.5 | 7.5 | 19.5 | 19.5 | 13.5 | 15.5 |
| of zone | | | | | | | |
| of | | | | | | | |
| inhibition | | | | | | | |

TABLE 2
MIC (marbofloxacin) of strain WSG1

| | MIC |
|---|---|
| WSG1 | 1.024 mg/ml |

It can be seen that WSG1 has good susceptibility to antibiotics.

### Experimental Example 3: Identification of Strains

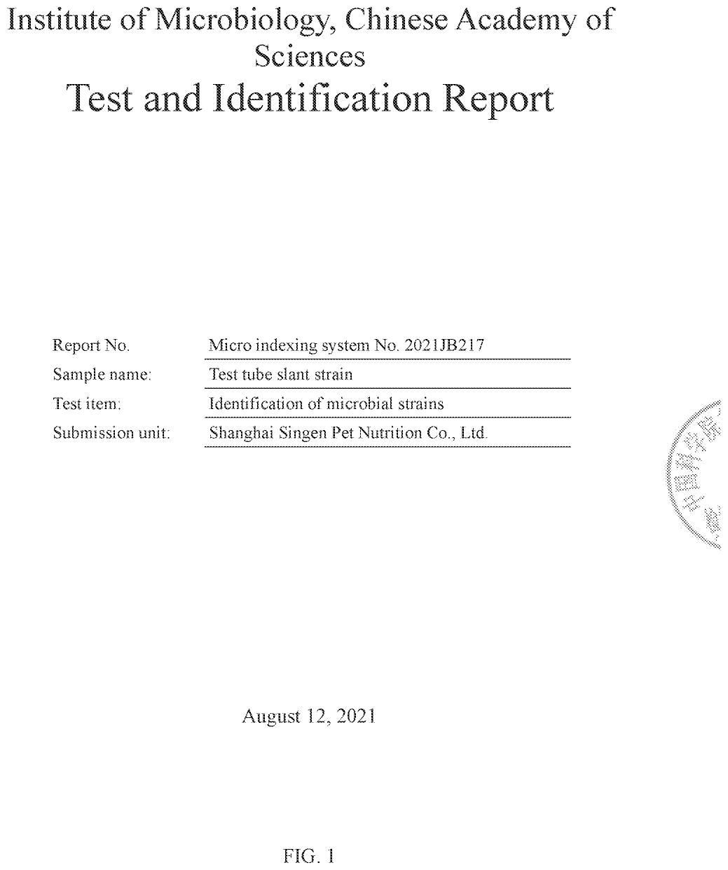

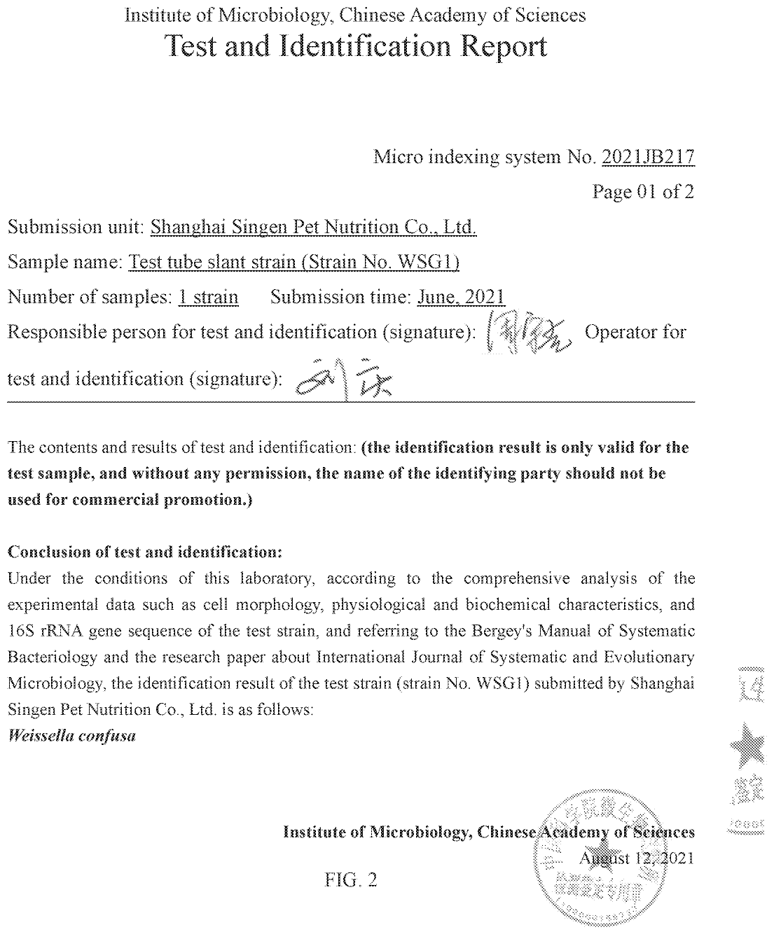

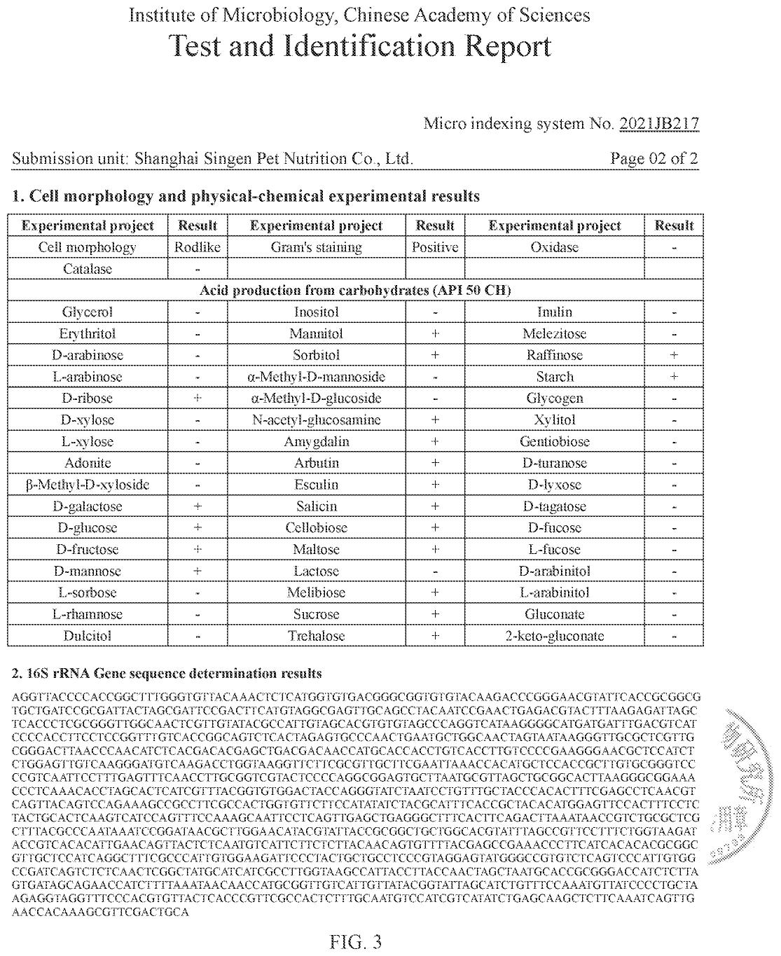

The samples were sent to the Institute of Microbiology, Chinese Academy of Sciences for identification, and identification reports were shown in FIGS. 1 to 3.

### Experimental Example 4: Evaluation on Acid Resistance and Bile Salt Resistance

The experimental process was as follows: through simulation of a gastrointestinal environment, the strain WSG1 was inoculated into culture media with different pH and bile salt concentrations, and the bacterial content variation was researched to determine the tolerance of the strain to acid and base (pH 2.0, pH 3.0, pH 4.0 and pH 5.0, and bile salt concentrations of 0.1%, 0.3% and 0.5%).

The experimental results were as follows: the survival rates of the strain WSG1 under different conditions were shown in Table 3.

TABLE 3
Survival rates of strain WSG1 under different conditions

| pH | | | | Bile salt concentration | | |
|---|---|---|---|---|---|---|
| 2 | 3 | 4 | 5 | 0.1% | 0.3% | 0.5% |
| 7.34 ± 2.10 | 5.75 ± 0.83 | 50.29 ± 2.22 | 81.59 ± 5.91 | 4.54 ± 0.55 | 7.88 ± 1.03 | 5.88 ± 0.68 |
| % | % | % | % | % | % | % |

It can be seen that WSG1 has the capacity to survive in the gastrointestinal environment.

### Experimental Example 5: Inhibitory Effect on Common Diarrhea-Causing Pathogenic Bacteria

The experimental process was as follows: indicator bacteria: Escherichia coli ATCC 25922, Salmonella typhimurium ATCC 14028, and Clostridium perfringens ATCC 13124. The strain WSG1 was cultured for 24 h and centrifuged to obtain a supernatant, and the supernatant was filtered through a 0.22 μm filter membrane, and the inhibitory effect of the supernatant on three indicator bacteria was researched using the punch hole method.

The experimental results were as follows: the diameters (mm) of the zones of inhibition of the supernatant of the strain WSG1 against three indicator bacteria were shown in Table 4, and the strain has a remarkable inhibitory effect.

TABLE 4
Diameters (mm) of zones of inhibition of supernatant
of strain WSG1 against three indicator bacteria

| Escherichia coli | Salmonella typhimurium | Clostridium perfringens |
|---|---|---|
| 9.5 | 7.5 | 10.5 |

### Experimental Example 6: Study on Digestion-Assisting Capacity-Protease-Producing Capacity

The experimental process was as follows: after overnight culture, the strain WSG1 was centrifuged and filtered to obtain a cell-free supernatant, the cell-free supernatant was inoculated into a 1% skimmed milk plate using the punch hole method, and the protease-producing capacity was determined by observing whether the cell-free supernatant produced a proteolytic ring or not, and if there was a protein dissolving phenomenon, then the strain was considered to have protein-degrading capacity.

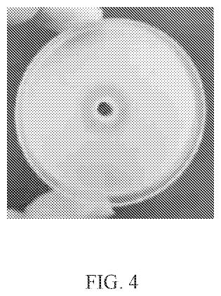

The experimental results were as follows: a proteolytic ring can be produced as shown in FIG. 4.

### Experimental Example 7: Oral Toxicity Study on Mice

The preparation of bacterial liquid was as follows: the strain WSG1 was inoculated into the MRS medium, and cultured for 24 h, followed by counting using the plate method to obtain a bacterial content reaching 4.75×10¹¹ CFU/mL.

After being adapted to raise for one week, 20 ICR mice aged 7 weeks were randomly divided into 2 groups, wherein the mice in the experimental group were orally administered with bacterial suspension diluted with sterile normal saline in an amount of 2.5×10⁹ CFU/Kg body weight/day, and the mice in the control group were orally administered with normal saline once with a dose of 1 mL. The mice were observed continuously for their activity, signs of toxicity and mortality within 10 days.

The experimental results were as follows: during the acute oral toxicity study of the strain WSG1, no mortality was exhibited in mice, the skin and hair, water drinking condition, physical activity and behavior patterns of the mice were normal, and no acute toxicity sign and relevant toxicity reaction were found.

The weight gain of the mice in the experimental group was reduced compared with the weight gain of the mice in the control group, but no significant difference existed.

### Experimental Example 8: Oral Toxicity Study on Dogs

The preparation of bacterial liquid was as follows: 90 μL of the strain WSG1 was pipetted using a 100 μL pipettor beside an alcohol burner, and poured into a 50 mL centrifuge tube, and then 30 mL of a 10% glucose solution was added to the centrifuge tube and mixed homogeneously.

Experimental animals were as follows: 20 laboratory dogs (4 young beagles, 4 adult beagles, 4 adult French bulldogs, 4 adult teddy dogs, and 4 adult Chinese rural dogs).

The dogs were equally divided into two groups (including a control group and an experimental group). The dogs in the control group were fed normally daily without restriction of water drinking. The dogs in the experimental group were fed normally daily without restriction of water drinking, and each dog was fed with WSG1 bacterial liquid prepared in step 1 at a dose of 0.5 mL/kg daily (finally equivalent to that each dog was fed with 1.5 μL of probiotic stock liquid at a dose of 7×10⁸ CFU/Kg body weight/day) in 2 portions. The feeding was continued for 10 days, and the feces of each dog were observed daily and scored according to the Waltham® Faeces Scoring System. Each dog was weighed before and after feeding.

The experimental results were as follows: the results of experimental groups were as follows:
(1) Young beagles: the feces of one beagle were not shaped well on the 1^{st} day and sticky on the 3^{rd} and 4^{th} days after feeding probiotics; the feces of another beagle were sticky and not shaped well on the 2^{nd} and 3^{rd} days, and looked well during the rest of time.
(2) Adult beagles: the feces of one beagle were soft on the 7^{th} day but basically shaped, and looked well during the rest of time.
(3) Adult Chinese rural dogs: no obvious abnormality in the feces was seen in the whole process.
(4) Adult teddy dogs: the feces of one teddy dog were sticky and not shaped well on the 6^{th} day, and looked well during the rest of time.
(5) Adult French bulldogs: the feces of one French bulldog were soft on the 3^{rd} day but basically shaped, and looked well during the rest of time.

The results of control groups were as follows:
(1) Young beagles: the feces of one beagle were sticky and not shaped well on the 1^{st} and 4^{th} days, soft but basically shaped on the 3^{rd} day, and looked well during the rest of time. The feces of another beagle were sticky and not shaped well on the 1^{st} and 3^{rd} days, and looked well during the rest of time.
(2) Adult beagles: the feces of one beagle were sticky and basically not shaped on the 1^{st} day, and looked well during the rest of time.
(3) Adult Chinese rural dogs: no obvious abnormality in the feces was seen in the whole process.
(4) Adult teddy dogs: no obvious abnormality in the feces was seen in the whole process.
(5) Adult French bulldogs: no obvious abnormality in the feces was seen in the whole process.

The data on the ninth day were analyzed using the Mann-Whitney U test with insignificant differences, as shown in Table 5, wherein the scores of the feces in both groups had no significant difference within ten days.

TABLE 5

| Experimental | | | | | | | | | | |
|---|---|---|---|---|---|---|---|---|---|---|
| group/Score | D1 | D2 | D3 | D4 | D5 | D6 | D7 | D8 | D9 | D10 |
| Young beagle 1 | 3 | 4 | 4 | 3.5 | 3 | 3 | 2.5 | 3 | 3 | 2.5 |
| Young beagle 2 | 4 | 3 | 4 | 4 | 3 | 3 | 3 | 3 | 2.5 | 3 |
| Adult beagle 1 | 3 | 2.5 | 2.5 | 3 | 3 | 3 | 3.5 | 3 | 3 | 2.5 |
| Adult beagle 2 | 2.5 | 3 | 3 | 3 | 2.5 | 2.5 | 3 | 3 | 2.5 | 2.5 |
| Adult French bulldog 1 | 2 | 3 | 2.5 | 2.5 | 2.5 | 2 | 3.5 | 3 | 2 | 2.5 |
| Adult French bulldog 2 | 3 | 3 | 3.5 | 3 | 3 | 2.5 | 2.5 | 3 | 3 | 2.5 |
| Adult Chinese rural | 3 | 2 | 3 | 2.5 | 2.5 | 2.5 | 2 | 3 | 2.5 | 2.5 |
| dog 1 | | | | | | | | | | |
| Adult Chinese rural | 3 | 2.5 | 3 | 3 | 2.5 | 3 | 2.5 | 3 | 2.5 | 2.5 |
| dog 2 | | | | | | | | | | |
| Adult teddy dog 1 | 3 | 2.5 | 2 | 3 | 3 | 3.5 | 2.5 | 2 | 2.5 | 2.5 |
| Adult teddy dog 2 | 3 | 3 | 2.5 | 3 | 3 | 2.5 | 2.5 | 3 | 2.5 | 2.5 |
| Control group/Score | D1 | D2 | D3 | D4 | D5 | D6 | D7 | D8 | D9 | D10 |
| Young beagle 1 | 3.5 | 3 | 3.5 | 3 | 3 | 3 | 2.5 | 3 | 3 | 3 |
| Young beagle 2 | 3.5 | 3 | 3 | 3.5 | 3 | 3 | 2.5 | 3 | 2.5 | 3 |
| Adult beagle 1 | 3 | 3 | 2.5 | 3 | 3 | 2.5 | 2.5 | 3 | 3 | 2.5 |
| Adult beagle 2 | 3.5 | 3 | 2.5 | 3 | 2.5 | 3 | 2 | 2 | 2.5 | 2.5 |
| Adult French bulldog 1 | 2.5 | 3 | 3 | 3 | 2.5 | 2.5 | 3 | 3 | 2.5 | 2.5 |
| Adult French bulldog 2 | 2.5 | 2.5 | 2 | 2.5 | 2 | 2.5 | 3 | 3 | 2.5 | 2.5 |
| Adult Chinese rural | 2.5 | 3 | 3 | 2.5 | 2.5 | 3 | 3 | 3 | 2.5 | 3 |
| dog 1 | | | | | | | | | | |
| Adult Chinese rural | 3 | 2.5 | 2.5 | 3 | 3 | 2.5 | 2.5 | 3 | 2.5 | 3 |
| dog 2 | | | | | | | | | | |
| Adult teddy dog 1 | 3 | 3 | 2.5 | 2.5 | 3 | 3 | 2.5 | 3 | 2.5 | 2.5 |
| Adult teddy dog 2 | 2.5 | 2.5 | 3 | 3 | 2.5 | 3 | 2.5 | 3 | 3 | 2.5 |

Body weights of these dogs in the experimental groups and the control groups increased over the experimental period, but there was no group-to-group difference, as shown in Table 6.

TABLE 6

| | |
|---|---|
| Body weight | Body weight |
| (kg) before | (kg) after |
| experiment | experiment |

Experimental group

| | |
|---|---|
| 8.23 | 9.67 |
| 8.08 | 9.32 |
| 13.54 | 14.03 |
| 12.56 | 12.58 |
| 9.02 | 9.95 |
| 8.16 | 8.68 |
| 7.04 | 7.88 |
| 6.78 | 6.94 |
| 5.24 | 5.96 |
| 7.83 | 8.21 |

Control group

| | |
|---|---|
| 8.39 | 8.94 |
| 7.74 | 8.54 |
| 14.32 | 15.12 |
| 16.36 | 17.62 |
| 9.67 | 10.83 |
| 10.06 | 10.48 |
| 7.48 | 8.62 |
| 9.18 | 9.32 |
| 8.92 | 9.64 |
| 11.36 | 11.98 |

Therefore, WSG1 is not toxic to dogs. According to the above examples, the present invention provides Weissella confusa WSG1 capable of promoting digestion and inhibiting common diarrhea-causing pathogenic bacteria, which has been proved to be safe by hemolytic evaluation and susceptibility evaluation, can survive in the gastrointestinal environment, has good susceptibility to antibiotics, has a remarkable inhibitory effect on common diarrhea-causing pathogenic bacteria, has digestion-assisting capacity, and is orally administered without toxicity. Therefore, a new available bacterial strain for the development of probiotics for maintaining the health capability of the intestinal tracts of canine organisms is provided herein.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. Weissella confusa, wherein the Weissella confusa is Weissella confusa WSG1 and preserved in the China General Microbiological Culture Collection Center (CGMCC) on Jun. 11, 2021 with a preservation number of CGMCC NO. 22697.

2. The Weissella confusa according to claim 1, wherein a nucleotide sequence of 16S IRNA of the Weissella confusa is shown in SEQ ID NO: 1.

3. A culture method for the Weissella confusa according to claim 1, comprising inoculating the Weissella confusa WSG1 into a culture medium for a culture, with an initial pH of the culture being 5.0 to 6.5, a temperature of the culture being 35° C. to 38° C., and a time of the culture being 15 h to 17 h; wherein
the culture medium comprises a carbon source and a nitrogen source, and a content of the carbon source and a content of the nitrogen source in the culture medium are independently 1.0% to 3.0%.

4. The culture method for the Weissella confusa according to claim 3, wherein the carbon source is one or more selected from the group consisting of glucose, sucrose, and starch.

5. The culture method for the Weissella confusa according to claim 4, wherein the nitrogen source is one or more selected from the group consisting of peptone, a beef extract, and a yeast powder.

6. A method of a use of the Weissella confusa according to claim 1 for a manufacturing of a medicament for assisting digestion.

7. The method of the use according to claim 6, wherein the medicament is a veterinary medicament.

8. The method of the use according to claim 7, wherein the veterinary medicament is a canine-specific medicament.

9. A method of a use of the Weissella confusa according to claim 1 for a manufacturing of a feed.

10. A method of a use of the Weissella confusa according to claim 1 for a manufacturing of a medicament for treating diarrhea.

11. The culture method for the Weissella confusa according to claim 3, wherein a nucleotide sequence of 16S rRNA of the Weissella confusa is shown in SEQ ID NO: 1.

12. The method of the use according to claim 6, wherein a nucleotide sequence of 16S IRNA of the Weissella confusa is shown in SEQ ID NO: 1.

13. The method of the use of the Weissella confusa according to claim 9, wherein a nucleotide sequence of 16S rRNA of the Weissella confusa is shown in SEQ ID NO: 1.

14. The method of the use of the Weissella confusa according to claim 10, wherein a nucleotide sequence of 16S rRNA of the Weissella confusa is shown in SEQ ID NO: 1.


SEQUENCE LISTING (1 sequence)

SEQ ID NO: 1 - DNA, 1429 bp, Artificial Sequence
  The sequence is synthesized.
aggttaccccaccggctttgggtgttacaaactctcatggtgtgacgggcggtgtgtacaagacccgggaacgtattcaccgcggcgtgctgatccgcgattactagcgattccgacttcatgtaggcgagttgcagcctacaatccgaactgagacgtactttaagagattagctcaccctcgcgggttggcaactcgttgtatacgccattgtagcacgtgtgtagcccaggtcataaggggcatgatgatttgacgtcatccccaccttcctccggtttgtcaccggcagtctcactagagtgcccaactgaatgctggcaactagtaataagggttgcgctcgttgcgggacttaacccaacatctcacgacacgagctgacgacaaccatgcaccacctgtcaccttgtccccgaagggaacgctccatctctggagttgtcaagggatgtcaagacctggtaaggttcttcgcgttgcttcgaattaaaccacatgctccaccgcttgtgcgggtccccgtcaattcctttgagtttcaaccttgcggtcgtactccccaggcggagtgcttaatgcgttagctgcggcacttaagggcggaaaccctcaaacacctagcactcatcgtttacggtgtggactaccagggtatctaatcctgtttgctacccacactttcgagcctcaacgtcagttacagtccagaaagccgccttcgccactggtgttcttccatatatctacgcatttcaccgctacacatggagttccactttcctctactgcactcaagtcatccagtttccaaagcaattcctcagttgagctgagggctttcacttcagacttaaataaccgtctgcgctcgctttacgcccaataaatccggataacgcttggaacatacgtattaccgcggctgctggcacgtatttagccgttcctttctggtaagataccgtcacacattgaacagttactctcaatgtcattcttctcttacaacagtgttttacgagccgaaacccttcatcacacacgcggcgttgctccatcaggctttcgcccattgtggaagattccctactgctgcctcccgtaggagtatgggccgtgtctcagtcccattgtggccgatcagtctctcaactcggctatgcatcatcgccttggtaagccattaccttaccaactagctaatgcaccgcgggaccatctcttagtgatagcagaaccatcttttaaataacaaccatgcggttgtcattgttatacggtattagcatctgtttccaaatgttatcccctgctaagaggtaggtttcccacgtgttactcacccgttcgccactctttgcaatgtccatcgtcatatctgagcaagctcttcaaatcagttgaaccacaaagcgttcgactgca